(19) United States
(12) Patent Application Publication
(10) Pub. No.: US 2007/0161599 A1
(43) Pub. Date: Jul. 12, 2007

(54) Composition for blocking hiv binding to dendritic cells and methods of use thereof

(75) Inventors: Richard Novak, Glenview, IL (US); Meghan Jendrysik, Washington, DC (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: The Board Of Trustees Of The University Of Illinois, Urbana, IL (US)

(21) Appl. No.: 10/558,496

(22) PCT Filed: May 28, 2004
(86) PCT No.: PCT/US04/17179

§ 371 Date: Feb. 21, 2007

Related U.S. Application Data
(60) Provisional application No. 60/474,078, filed on May 28, 2003.

(51) Int. Cl. A61K 31/715; C12P 19/04

(52) U.S. Cl. 514/54; 536/123

(57) ABSTRACT

Compounds and compositions for inhibiting binding between dendritic cell-specific ICAM-3 grabbing non-integrin (DC-SIGN) and human immunodeficiency virus (HIV).

### PRIORITY INFORMATION

This application claims priority to U.S. Provisional Application 60/474,078, filed May 28, 2003.

### BACKGROUND OF THE INVENTION

Dendritic Cell-Specific ICAM-3 Grabbing Non-integrin (DC-SIGN) is a 44 kDa C-type lectin expressed on the surface of Dendritic Cells (DC), particularly immature DC. It is found on dermal DC and DC-like cells in the lamina propria of the mucosae of the rectum, cervix, and uterus. DC-SIGN contains a carbohydrate recognition domain (CRD) that is specific for mannose and dependent on calcium ions for binding, and separate binding sites for gp 120 (the HIV envelope glycoprotein) and the Intercellular Adhesion Molecule 3 (ICAM-3). ICAM-3 is a co-stimulatory adhesion molecule expressed at high levels on resting T cells, and is normally bound by Lymphocyte Function-Associated antigen Type 1 (LFA-1). DC-SIGN mediates transient adhesion to T cells via this binding. The HIV surface glycoprotein gp120 is responsible for binding to suitable host cell receptors, typically CD4.

The primary role of DC-SIGN is in the formation of the “Immunological Synapse”. By forming this synapse, antigen-presenting cells (such as DC) are brought into very close contact with T cells, by means of several distinct receptor-ligand pairs, including CD48 and CD2, CD80 and CD28, and MHC (with bound antigen) and TCR. DC-SIGN may also be involved in this interaction through binding ICAM-3 expressed on the T cell. The immunological synapse increases the activation of the T cell, by efficient antigen presentation coupled with stimulatory cytokines. This is the first step in the formation of the immunological synapse. In this case, the synapse could also spread virus more efficiently as well. This could be an important step in establishing HIV infection, the virus “hitching a ride” on DC and traveling from the mucosa to the lymph nodes.

Geijtenbeek et al. originally described DC-SIGN as an immune mediator. They found that the amino acid sequences of two DC-SIGN peptides matched those of a membrane-bound mannose-specific lectin that had been proposed as an HIV gp120 receptor in 1992, cloned from a human placental cDNA library, but never fully identified. Based on these sequence similarities, they decided to investigate the DC-SIGN's ability to bind gp120. DC-SIGN (and homologues) not only bind HIV, but enhance trans-infection of T cells (in vitro). After binding to DC-SIGN, HIV is internalized to a vesicle within the cell, where it maintains its infectivity. Based on these observations, it has been suggested that DC-SIGN and dendritic cells play a role in establishing HIV infection in the vaginal mucosa, and possibly at other mucosal surfaces. The proposed model is that once HIV has bound to the dendritic cell via DC-SIGN, it is carried by the cell to the lymph node, where the virion is presented to a large number of T cells not for antigen recognition but for infection of these cells. Through this same interaction, the DC-SIGN/ICAM-3 interaction results in T-cell activation, providing an ideal target cell for productive viral replication. This “Trojan Horse” mechanism of transmission has been well established for other lentiviruses, such as maedi-visna virus in sheep.

Thus, there is a need for compounds and compositions that inhibit the binding of HIV to dendritic cells, thereby preventing the “Trojan Horse” mechanism of transmission.

### SUMMARY OF THE INVENTION

The present invention is directed to an agent that inhibits the binding of HIV to DC. This agent may be used for to provide a method to prevent HIV-1 or HIV-2 infection. In certain embodiments, the agent may be used to prevent vaginal and anal transmission of HIV-1 or HIV-2 during sexual intercourse. Other embodiments employ this agent to prevent vaginal transmission of HIV-1 or HIV-2 during childbirth. Still other embodiments employ the compositions of the invention to prevent, reduce or otherwise inhibit the transmission of HIV-1 or HIV-2 to a child during breast feeding.

In particular aspects, the present invention is directed to a compound isolated from a cervicovaginal lavage that inhibits binding between dendritic cell-specific ICAM-3 grabbing non-integrin (DC-SIGN) and human immunodeficiency virus (HIV), said compound comprising a carbohydrate moeity; lacking a terminal mannose residue; having a molecular weight of greater than 100 kDa; retaining function after ten minutes at 95° C.; binding to DC-SIGN; and inhibiting binding of HIV to DC-SIGN. The compound has been demonstrated as being resistant to digestion by proteases. For example, it has been shown to be resistant to trypsin digestion. The compound is however susceptible to periodate oxidation. The compound also may be characterized as an agent that binds to DC-SIGN in a calcium-dependent manner. The composition described herein may preferably formulated as a pharmaceutical composition. Such a pharmaceutical composition may comprise the compound derived from genital tract of women alone or in combination with a pharmaceutically-acceptable carrier. The compound also may be provided with additional active agents that prevent HIV-1 and/or HIV transmission. Preferably, the composition is formulated for vaginal administration. Alternatively, the composition is formulated for rectal administration.

The present invention also is directed to a method of inhibiting binding of HIV to DC-SIGN, the method comprising contacting a cell expressing DC-SIGN on its surface with a compound described herein in an amount effective to inhibit HIV binding to DC-SIGN. The cell may be contacted in vitro. Alternatively, the cell is contacted in vivo. The composition may be is administered to a patient at risk of exposure to HIV. The composition may be administered to a patient subsequent to exposure to HIV at a location in the patient. In specific embodiments, the composition is administered to the location of exposure.

Also contemplated is a method of isolating a microbe that produces a compound of the present invention comprising testing for a presence of a compound of the invention in vaginal fluid of a woman; isolating an organism from the vaginal fluid; and testing the organism for production of the compound.

Kits comprising the compounds of the invention also are contemplated.

Other aspects of the invention describe methods of determining the structure of a compound of the invention comprising isolating the compound from a cervicovaginal lavage and determining the structure of the carbohydrate.

Also provided is a purified and isolated compound which is a carbohydrate which, in nature, is produced in the genital tracts of women that have been exposed to the semen of an HIV positive sexual partner but are HIV negative wherein the compound has a molecular weight greater than 100 kDa, is resistant to trypsin digestion, is resistant to temperatures greater than 80° C., and is an inhibitor of HIV binding to DC-SIGN. In specific embodiments, the compound is formulated in a combination with a pharmaceutically acceptable carrier or diluent. In other embodiments, the compound is formulated into a composition for topical administration.

The present invention also provides a method of preventing transmission of HIV-1 infection or HIV-2 infection which comprises locally administering to an appropriate region of a human body a pharmaceutically effective anti-HIV-1 or anti-HIV-2 amount of the inhibitor from a CVL described herein or a pharmaceutically acceptable salt thereof. Such method is intended to prevent transmission of HIV infection, for example, during close bodily contact between two individuals under conditions which would generally favor HIV transmission, for example, during sexual intercourse or during childbirth.

The phrase “administration to an appropriate region of the human body” includes, for example, application of inhibitor to regions of the human body which come into close contact with another human body, for example, application to the male or female genitalia if the method is intended to prevent transmission during sexual intercourse, and application to the vagina or to a baby's epidermis if the method is intended to prevent transmission during childbirth.

The term “locally administrating” includes any method of administration in which the activity of the inhibitor identified herein is substantially confined to the region of the human body to which it is applied, for example, vaginal, rectal or topical administration.

The present invention thus provides a method of preventing vaginal transmission of HIV-1 or HIV-2, either during sexual intercourse or during childbirth (vaginal delivery), by vaginal administration, such as by administering a cream, ointment, lotion, jelly, solution, emulsion or foam formulation containing a pharmaceutically effective amount of a an inhibitory compound described herein, wherein the amount is effective to inhibit the binding of HIV to DC-SIGN.

The present invention also therefore relates to a method of preventing transmission of HIV-1 or HIV-2 in a newborn baby by topically administering to the baby soon after childbirth an effective amount of a CVL derived inhibitor described herein, either alone or in combination with a carrier, wherein the amount is effective to inhibit the binding of HIV to DC-SIGN.

The present invention is also directed to a contraceptive device (for example, a male or female condom, a contraceptive diaphragm or a contraceptive sponge, for example, a polyurethane foam sponge), for the prevention of pregnancy, wherein the device has applied thereto an anti-HIV-1 or anti-HIV-2 effective amount of a compound described herein or a pharmaceutically acceptable salt thereof.

The present invention is further directed to a pessary or tampon for vaginal administration, wherein the tampon or pessary comprises, as an active ingredient, a pharmaceutically effective amount of a compound described herein or a pharmaceutically acceptable salt thereof and one or more pharmaceutically acceptable carriers or excipients, wherein said amount is sufficient to inhibit the binding of HIV to DC-SIGN.

The present invention further relates to a pharmaceutical composition for topical administration comprising an effective amount of a CVL-derived compound described herein, or a pharmaceutically acceptable salt thereof and at least one pharmaceutically acceptable topical carrier or excipient, to form an ointment, cream, gel, lotion, paste, jelly, spray or foam.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1 shows the experimental design used in the methods described herein.

FIG. 2. shows that CVLs inhibit BAL and MN equally.

FIG. 3 shows inhibition of HIV binding by CVL.

FIG. 4 shows that the inhibition of the binding is dose-dependent.

FIG. 5 shows that the inhibition is not due to cytotoxicity.

FIG. 6 shows that CVL does not inhibit viral binding or growth in PBL.

FIG. 7 shows the distribution of IC₅₀S in fractions of CVL.

FIG. 8 shows the IC₅₀S at baseline.

FIG. 9A through 9C shows the effect of treatment of BV.

FIG. 10 shows the results of a primary dendritic cell binding assay (MDDC).

FIG. 11 shows the results of binding in primary isolates.

### DETAILED DESCRIPTION OF THE INVENTION

The present invention provides compositions and methods for inhibiting the binding of DC-SIGN and HIV. In one aspect, the composition is a microbicide (viricide) that can be used by individuals to protect themselves from infection with HIV. Because the compound of the present invention is highly potent in very low concentrations, small amounts may be enough to confer protection. The compound or composition may be applied vaginally or rectally and formulated for such application method. As used herein, a microbicide need not destroy virus in the strictest sense, but rather the composition can block binding to a receptor involved with HIV transmission.

In another aspect of the invention, the composition is used to treat an HIV infected individual. Interference with gp120/DC-SIGN interactions in lymphatic tissue can have an antiviral effect. In this case, the composition would slow disease progression in infected individuals. In preferred methods of treating HIV infected individuals, compositions containing an effective amount of DC-SIGN/HIV-binding-inhibiting compound of the present invention are administered. Preferably, such compositions are formulated for oral, nasal, interperitoneal, or intravenous administration.

The invention provides a method of isolating a compound or composition that inhibits the binding between DC-SIGN and HIV. The compound that inhibits the binding between DC-SIGN and HIV is a natural substance found in the genital tract of some women. Thus, in one aspect of the invention the compound is isolated from the genital tract of such a woman.

The compound may be produced by a microbe, such as a virus, yeast, or bacterium. The microbe may be naturally occurring or recombinant. In one aspect of the invention, the compound is isolated from a culture of microbes. The isolation can involve isolating the compound from the growth medium or involve lysing the microbe and isolating the compound from the lysate.

In certain aspects of the invention, a microbe producing a compound or composition of the present invention is administered to an individual to produce an engineered flora. This engineered flora confers protection from HIV infection. Such a method of producing an engineered flora would be particularly useful in underdeveloped countries, where medical resources and personnel are scarce.

Preferred compounds or compositions of the present invention have one or more of the following characteristics:

1) inhibits the binding of HIV (primary or laboratory strains) to DC-SIGN;

2) molecular weight of greater than 100 kDA;

3) lacks binding to DEAE Cephacel affinity column;

4) lacks binding to SP sepharose;

5) lacks terminal mannose residues;

6) retains activity after subjected to a Staphylococcus protein A column;

7) lacks binding to Jacalin-agarose beads;

8) retains function after being subjected to trypsin digest;

9) binds to DC-SIGN-expressing cells;

10) binds to DC-SIGN agarose matrix;

11) susceptible to periodate oxidation; and

12) stable after ten minutes at 95° C.

In a preferred embodiment, the compound or composition retains characteristic 1) above and one, two, three, four, five, six, seven, eight, nine, ten, or eleven of the additional itemized characteristics. In one embodiment, the compound or composition comprises a carbohydrate or a low molecular weight polypeptide with carbohydrate side chains. This compound or composition binds to DC-SIGN in an energy-independent, calcium-dependent manner. As indicated above, the compound is retains its function after being subjected to trypsin digestion. As such, it is resistant to proteolysis. In addition to trypsin, other typical proteolysis reactions can be performed with the use of an enzyme e.g., papain, lyc-C protease or pepsin to yield cleavage of the antibody at the hinge region.

In another aspect, the present invention provides a method of isolating a compound or composition of the present invention comprising the steps of contacting a matrix comprising DC-SIGN with a composition comprising the compound or composition of the present invention and eluting the compound or composition with a chelator such as EDTA.

The compounds of the present invention are isolated from the female genital tract of women that are at high exposure risk for HIV infection but are HIV-negative. Typically, such women have a history of HIV-positive sexual partners and yet have remained HIV negative. Cervicovaginal lavages of such “high risk” women produce an inhibitory agent that blocks primary isolates of HIV from binding to DC-SIGN. This compound may contribute to protection from HIV transmission. The compound blocks binding of R5 primary isolates to DC. The compound has a molecular weight of greater than 100 kDa, is stable after being subjected to 95° C. for 10 minutes but is susceptible to periodate oxidation.

Methods of performing cervicovaginal lavages to isolate the inhibitory compound are known to those of skill in the art. Typically, in such procedures, following the introduction of a speculum, a standardized 60-second lavage is performed with 10 ml of normal saline. The vaginal secretions are then typically centrifuged to remove debris. In the present studies the preparation is heat inactivated for complement, after which the lavages are centrifuged at e.g., 1,000×g for 10 min. Other methods of performing CVLs have been previously described (see e.g., Belec et al., Clin. Diagn. Lab. Immunol. 2:57-61, 1995). Lavage samples may be confirmed to be devoid of a significant amount of contaminating blood by measuring traces of hemoglobin using second derivative spectrophotometry (Sanderink et al., Clin. Chim. Acta 28:65-73, 1985; Si-Mohammed et al., J. Infect. Dis. 182:112-122, 2000). The lavages also may be filtered using a filter with a pore size retains compounds that have a molecular weight of greater than 80 kDa because it was shown herein that the majority of the CVL inhibitory activity of the present invention was retained by filters that retained compounds greater than 100 kDa. As discussed below it is likely that the agent is a carbohydrate such as a polysaccharide which does not contain a terminal mannose moiety.

Following isolation of the compound, the structure of the compound may be determined. Although not necessary in order to make and use the compounds, compositions, and methods of the invention, knowledge of the components and structure of the polysaccharide would facilitate the de novo synthesis of the compound and the design of structural variants that retain function or structural variants with increased inhibitory activity.

It is contemplated that the compound comprises a carbohydrate or polysaccharide component. Sensitive methods of sequencing polysaccharides exist. For example, U.S. Pat. No. 6,597,996 describes a matrix-assisted laser desorption ionization mass spectrometry (MALDI-MS) technique that is accurate to <1 dalton and sensitive down to 100 fmol of material. (See also Venkataraman et al., Science 286:537-542 and U.S. Patent Application Publication No. 20040091472.) Briefly, the technique involves the reduction in size of starting oligonucleotides into smaller fragments through the successive use of various chemical and enzymatic degradation methods. The degradation products are subsequently analized by MALDI-MS to determine the length of the saccharide and the number of acetates and sulfates contained therein. The results are then compiled to converge to a single unique sequence (structure).

Others describing methods of determining the structure of polysaccharides include Callawaert et al. (Glycobiology 11(4):275-281, 2001), Sagi et al. (J. Am. Soc. Mass. Spectrom. 13:1138-1148, 2002), and Thanawiroon et al. (J. Biol. Chem. 279(4):2608-15, 2004).

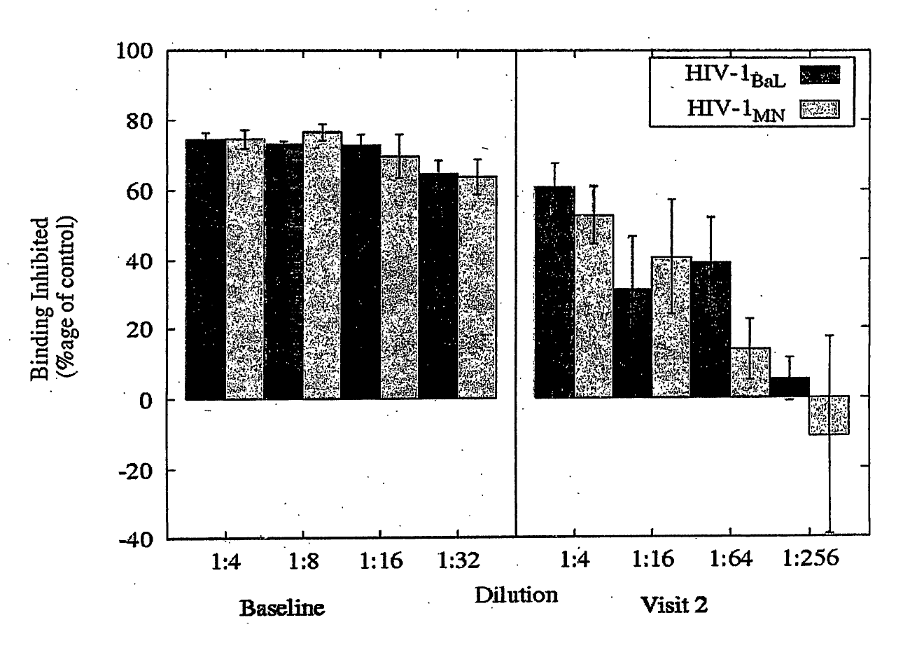

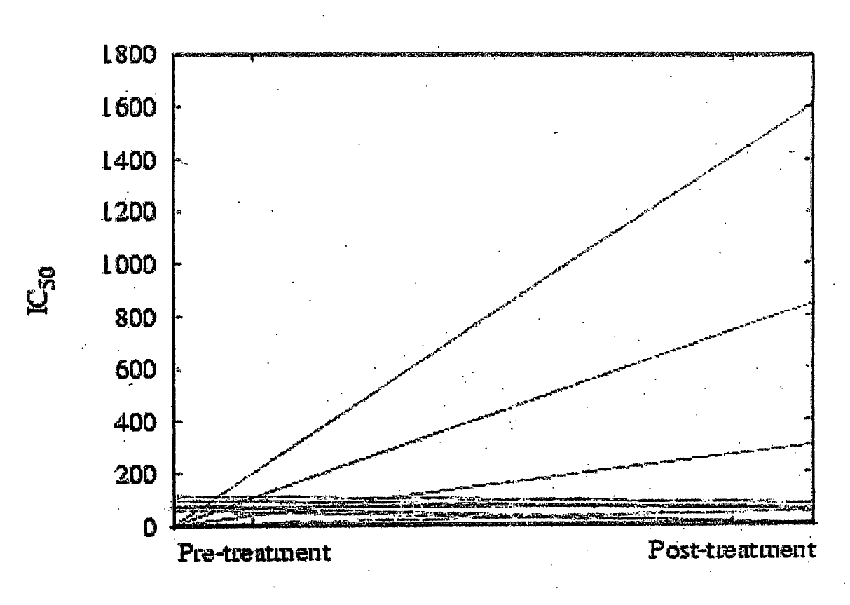

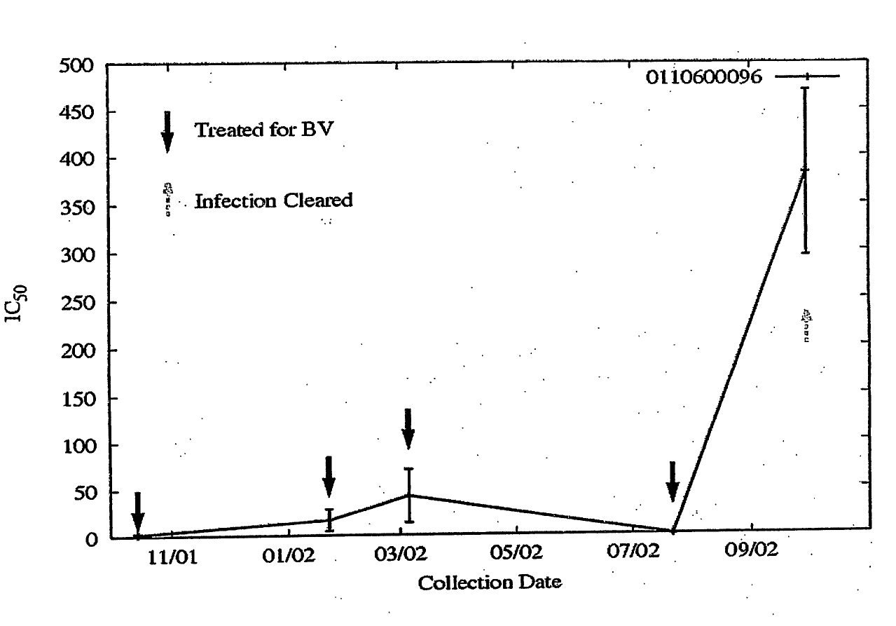

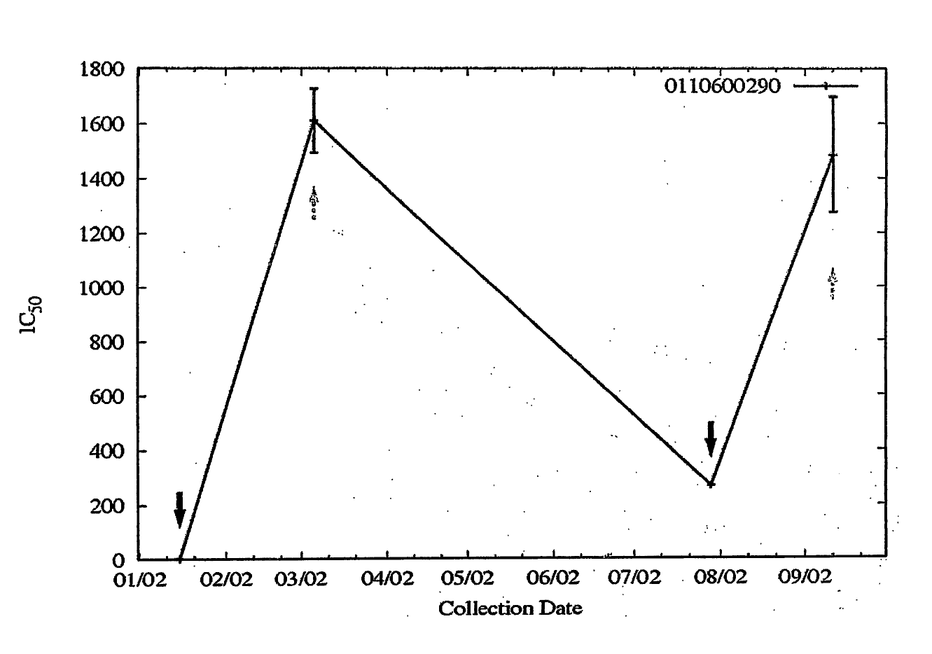

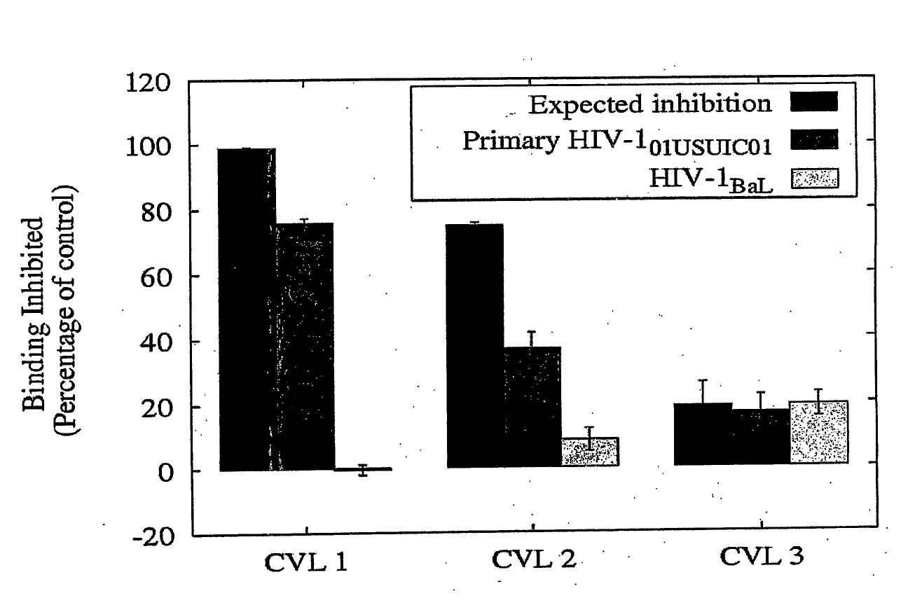

The inhibitor is tested for its inhibitory activity by determining its effect on the binding DC cells to HIV particles. In examples shown herein this activity is demonstrated using the binding of DC-SIGN to HIV-1_{BAL}. The data from such experiments is shown in FIGS. 2 to 11. The binding of HIV virions to DC cells may be determined using any assay known to those of skill in the art. In exemplary embodiments shown herein, the bound HIV was assessed by performing a p24 assay on the cellular lysates and the percentage of inhibition of binding was calculated as the percentage of HIV bound by THP DC-SIGN cells exposed to mock CVL composed of saline. The data in FIGS. 2 to 11 clearly demonstrate that the genital tracts of some women at a high risk for high infection contain a potent inhibitor of DC-HIV binding.

DC-SIGN, a type II transmembrane mannose-binding C-type lectin, is important in the function of DC, both in mediating naive T cell interactions through ICAM-3 and as a rolling receptor that mediates the DC-specific ICAM-2-dependent migration processes. It can be used by viral and bacterial pathogens including Human Immunodeficiency Virus (HIV), HCV, Ebola Virus, CMV and Mycobacterium tuberculosis to facilitate infection. Both DC-SIGN and DC-SIGNR can act either in cis, by concentrating virus on target cells, or in trans, by transmission of bound virus to a target cell expressing appropriate entry receptors. The isolated inhibitor may readily be formulated into pharmaceutical or prophylactic compositions that can be used to treat or prevent HIV transmission and/or the transmission of other viral entities. As the CVL-isolated inhibitor inhibits the interaction of DC-SIGN with viral particles, it is contemplated that such compositions will not only be useful in protection of an uninfected individual from infection upon exposure to HIV where the uninfected person uses the compositions, but will also be useful as prophylactics for use by the infected individual. This may be particularly important in preventing or reducing the rate of vertical transmission of HIV from HIV-positive women to infants during birth. For example, it has been suggested that an important feature in preventing transmission of HIV from mother to child is achieving a non-detectable viral load at the time of delivery. The compositions of the invention inhibit the binding of HIV to DC-SIGN. As such, these compositions may be formulated as creams, lotions or washes that could be used to bathe the woman at the time of delivery to prevent or reduce the transmission of HIV from mother to child. Further, it is noted that after birth HIV transmission from mother to child has occurred as a result of breast feeding. It is contemplated that the formulations of the present invention may be prepared for topical application to the inside of an infant's mouth to prevent or reduce the transmission of virus to the child during feeding.

Additional topical formulations particularly contemplated are formulations suitable for vaginal administration, which may be presented as pessaries, tampons, creams, gels, pastes, jelly, foams or sprays or aqueous or oily suspensions., solutions or emulsions (liquid formulations) containing in addition to the active ingredient, such carriers as are known in the art to be appropriate. These formulations are useful to protect not only against sexual transmission of HIV, but also to prevent infection of a baby during passage through the birth canal. Thus the vaginal administration can take place prior to sexual intercourse, during sexual intercourse, immediately prior to childbirth or during childbirth.

As a vaginal formulation, the active ingredient may be used in conjunction with a spermicide and may be employed with condoms, diaphragms, sponges or other contraceptive devices.

Still further compositions contemplated are washes for use as dentifrices mouthwashes and the like to be used in the event of oral exposure to HIV-1 or HIV-2. The compositions also may be formulated as washes or swabs for cleaning a wound that may be infected with HIV or may be at a risk of being infected with HIV.

The CVL inhibitor compositions according to the invention will generally comprise a vehicle to act as a dilutent, dispersant or carrier for the CVL inhibitor active ingredients in the composition, so as to facilitate the distribution of the inhibitor when the composition is applied to a given area. Preferably the vehicle is cosmetically and/or pharmaceutically acceptable. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions, its use in therapeutic compositions is contemplated. Supplementary active ingredients also can be incorporated into the compositions. For example, one will generally desire to employ appropriate salts and buffers to render the CVL inhibitor compositions stable and allow for uptake of the compositions at the target site. The isolated CVL inhibitor composition may be provided in a lyophilized form to be reconstituted prior to administration. Buffers and solutions for the reconstitution of the compositions may be provided along with the pharmaceutical formulation to produce aqueous compositions of the present invention for administration.

Vehicles other than water can include liquid or solid emollients, solvents, humectants, thickeners and powders typically found in cosmetic formulations. Examples of each of these types of vehicle, which can be used singly or as mixtures of one or more vehicles, are as follows:

Emollients, such as stearyl alcohol, glyceryl monoricinoleate, glyceryl monostearate, mink oil, cetyl alcohol, isopropyl isostearate, stearic acid, isobutyl palmirate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, tallow, lard, olive oil, palm kernel oil, rapeseed oil, safflower seed oil, evening primrose oil, soybean oil, sunflower seed oil, avocado oil, olive oil, sesame seed oil, coconut oil, arachis oil, castor oil, acetylated lanolin alcohols, petroleum jelly, mineral oil, butyl myristate, isostearic acid, palmitatic acid, isopropyl linoleate, lauryl lactate, myristyl lactate, decyl oleate, myristyl myristate;

Propellants, such as air, propane, butane, isobutane, dimethyl ether, carbon dioxide, nitrous oxide;

Solvents, such as squalene, squalane, ethyl alcohol, methylene chloride, isopropanol, acetone, ethylene glycol monoethyl ether, diethylene glycol monobutyl ether, diethylene glycol monoethyl ether, dimethyl sulphoxide, dimethyl formamide, tetrahydrofuran;

Humectants, such as polyhydric alcohols including glycerol, polyalkylene glycols and alkylene polyols and their derivatives, including propylene glycol, dipropylene glycol polypropylene glycol, polyethylene glycol and derivatives thereof, sorbitol, hydroxysorbitol, 1,3-butylene glycol, 1,2,6-hexanetriol, ethoxylated glycerol, propoxylated glycerol and mixtures thereof.

Powders, such as chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate.

The vehicle will usually form from 10 to 99.9%, preferably from 50 to 99% by weight of the emulsion, and can, in the absence of other adjuncts, form the balance of the composition.

A particularly convenient form of the composition according to the invention is an emulsion, in which case an oil or oily material will normally be present, together with an emulsifier to provide either a water-in-oil emulsion or an oil-in-water emulsion, depending largely on the average hydrophillic-lyophilic balance of the emulsifier employed. Such emulsions may provide useful barrier-forming virucides when combined with the CVL inhibitors of the present invention

Compositions according to the invention can optionally comprise one or more oils or other materials having the properties of an oil. Examples of suitable oils include mineral oil and vegetable oils, and oil materials, such as those already proposed herein as emollients. Other oils or oily materials include silicone oils, both volatile and non-volatile, such as polydimethyl siloxanes. The oil or oily material, when present for the purposes for forming an emulsion, will normally form up to 90%, preferably from 10 to 80% by volume of the composition.

Compositions according to the invention may also optionally comprise one or more emulsifiers, the choice of which will normally determine whether a water-in-oil or an oil-in-water emulsion is formed. Particular reference is made to e.g., U.S. Pat. No. 5,545,402 which describes various methods and commercially available components of such emulsifiers.

In addition to topical formulations, the CVL inhibitors also may be formulated for oral administration e.g., as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It will preferably be stable under the conditions of manufacture and storage and be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. As such, it is contemplated that the CVL inhibitor composition isolated by filtering through a filter that retains moieties that have a molecular weight larger than 100 kDa may directly be freeze-dried to produce such a powder.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkeys, ducks and geese. Models that mimic HIV infection will be particularly useful for testing and determining optimal amounts of CVL inhibitor composition to be administered.

### EXAMPLES

Embodiments of the present invention will be described with reference to the following examples, which are presented for illustrative purposes only and are not intended to limit the scope of the invention.

### Example 1 Materials and Methods

The present example provides exemplary experimental protocols employed to generate certain of the data described herein.

a. Participants and Sample Collection

Women were recruited in accordance with UIC's IRB. At the initial screening, candidates filled out a detailed questionnaire e of their risk behavior, personal habits (such as drug and alcohol use), and medical history. Blood was drawn for HIV serology. Women meeting the criteria (see Results) returned to the clinic for physical examination, at which time cervico-vaginal lavages (CVLs) were collected. Collection was carried out as follows: 10 mL of sterile saline were ejected against those of the cervix, and collected back in the same pipette. Lavages were centrifuged, and the supernatant was stored at −80° C. until used. At that time, aliquots were heat inactivated (54° C., 15 minutes) and filtered (0.22 μm).

b. Tests Run on CVLs

Semen antigen tests were performed using OneStep Abacard p30 test kits according to manufacturer's instructions (Abacus Diagnostics, Inc. West Hills, Calif.). Total cell number was determined by a manual count. White blood cell/red blood cell counts and the assessment of presence of sexually transmitted diseases were determined using standard methods.

c. Cell Lines and Viral Stocks

THP DC-SIGN and matched parenteral THP cells were kindly provided by Dan Littman (New York University). HIV-1_{BaL} and HIV-1_{MN} were obtained from the AIDS Reference and Reagent Program, Division of AIDS, NIAID, NIH. HIV-1_{BaL} was propagated in normal donor PBMC, HIV-1_{MN} was propagated in CEM-SS cells

d. Primary Viral Isolates

Five primary viral isolates were collected from acutely infected patients in our clinic. Patient PBMCs were co-cultured with normal donor PBMCs that had been PHA-stimulated for three days. The same healthy donor was used for all viral isolations. The patient genders and routes of transmission for the various isolates were as follows: HIV-1_{101USUIC014} (female) heterosexual exposure HIV-1_{931USUIC01} (female) injection drug use HIV-1_{931USUIC02} (male) homosexual exposure HIV-1_{931USUIC03} (male) injection drug user HIV-1_{931USUIC01} (male) unknown; risk factors included both sex with men and injection drug use. Viral tropisms were determined based on the ability to infect transfected indicator cells (Ghost R3/X4/R5, Ghost X4, and Ghost Hi5 cell lines obtained from the AIDS Reference and Reagent Program, Division of AIDS, NIAID, NIH).

e. Binding Assays

THP and THP DC-SIGN cells were resuspended at a concentration of 20×10⁶/mL, and dispensed to microfuge tubes (final concentration 2×10⁶ cells/tube). Serial dilutions of CVLs in D-PBS were prepared, and 500 μL of CVL dilutions were added to respective tubes. HIV (10,000 picograms in 500 μL) was allowed to bind to the cells during an hour long incubation at 37° C. Unbound HIV was removed by washing three times in wash medium (RPMI 1640 supplemented with 1% each of HEPES buffer and HI-FBS, plus 50 U/mL of penicillin/streptomycin). The washed cells were lysed in wash medium plus 10% Triton X-10, and the p24 antigen concentration was determined by ELISA. A “mock CVL” of D-PBS served as control. Background/non-specific HIV binding was determined by exposing untransfected THP cells to the mock CVL and virus. The results were interpreted as a percentage of HIV blocked compared to the control. The formula used to calculate binding inhibition is:
% Inhibition=100−(([p24]ₜₑₛₜ−[p24]_{background})/([p24]_{control}−[p24]_{background}))*100

The concentrations of cells and virus were optimized to yield a maximum HIV binding in the range of 5K to 8K pg of p24 antigen per milliliter, which corresponds to the upper limit of detection in the ELISA kit used (AIDS Vaccine Program, National Cancer Institute, Frederick, Md.).

f. Definition of IC₅₀

The ability of a CVL to inhibit HIV-DC-SIGN interaction is reported here as inhibitory concentration (50%), or “IC₅₀.” All CVLs were tested for inhibiting ability for at least two dilutions, typically 1:10 and 1:100 (or 1:50). If HIV-DC-SIGN binding was fully inhibited at 1:100, further dilutions were tested, until less than 50% inhibition was observed. The results of these assays were plotted (dilution v. % inhibition), and the slope of the line was used to predict at what dilution 50% of the virus would be blocked. The IC₅₀ value reported is the inverse of that dilution. In the event of a positive slope (which would indicate a negative IC₅₀), the result was interpreted as “no activity and assigned an IC₅₀ of 0.

g. Primary Dendritic Cells

Mononuclear-derived dendritic cells (MDDC) were matured from normal donor macrophages. CD14⁺ cells were positively isolated from PBMCs obtained as described above using the MACS CD14 microbeads and separated by AutoMACS (protocol Possel)(Miltenyi Biotec, Auburn, Calif.). CD14⁺ cells were immediately cultured in AIM-V medium supplemented with 1000 U/mL of GM-CSF (Immunex Corporation, Seattle, Wash.) and rIL4 (R&D Systems, Incorporated, Minneapolis, Minn.). The cells were fed on day 4, and harvested on day 7. By this time, the majority of cells had differentiated into DC. CD14⁺ cells were cryopreserved following an established protocol (for use as target cells in Transfer assays).

h. Transinfection Assay

Cryopreserved CD14+ PBMC were activated in the presence of rIL-2 and PHA for three days as described above. MDDC were incubated at 37° C. for 30 minutes in the presence of CVL or mock CVL (DPBS), and gently agitated at least twice during this time. HIV stock (500 microliters, 40,000 pg/mL) was then added, and the cells were then incubated 2 hours, gently agitating every 10 minutes. The cells were then washed to remove all unbound virus, resuspended, and added to wells already containing target lymphocytes at a ratio of approximately 1:20 MDDC:PBL. The transfer infections were incubated under standard conditions, and sampled at days 3 and 7. Viral growth was quantified by p24 ELISA.

i. Description of Study Cohorts

Participants in both cohorts had to be HIV⁻ (serology was performed at initial screening). Study participants were grouped according to the following self-reported risk behaviors.

Women enrolled in the high-risk cohort had to meet at least 2 of the following criteria:
crack use in the last 6 months
exchange of sex for money, drugs, or shelter in the last 6 months
at least 5 sexual partners in the last 6 months
history of sexually transmitted disease in the last year
OR
sexual relations with an HIV+ man

Women in the low-risk cohort had to meet the following criteria:
never used crack
never exchanged sex for money, drugs, or shelter
no more than one sexual partner in the last 6 months
no more than 5 sexual partners in the last 5 years
no history of STDs

A total of 32 low-risk and 63 high-risk women were enrolled.

Study visits occurred at enrollment (baseline) and six months. Participants were evaluated for bacterial vaginosis (BV), chlamydia, gonorrhea, and Trichomonas vaginalis at both visits, and any active infections were treated. If the infection was still active at a two-week follow-up visit (or if a new infection was diagnosed), further treatment was provided, and another follow-up scheduled. This process was repeated until the participant was cleared of all infections. Over 20% of the women in the high-risk cohort presented an active vaginal infection at enrollment.

### Example 2 Isolation and Characterization of a Compound that Inhibits Binding of HIV to DC-SIGN

As discussed herein; the vaginal fluids of some women can inhibit binding between DC-SIGN and HIV. Cervicovaginal lavage (CVL) specimens were screened from high risk, and low risk women. All subjects were HIV seronegative and free of other sexually transmitted diseases.

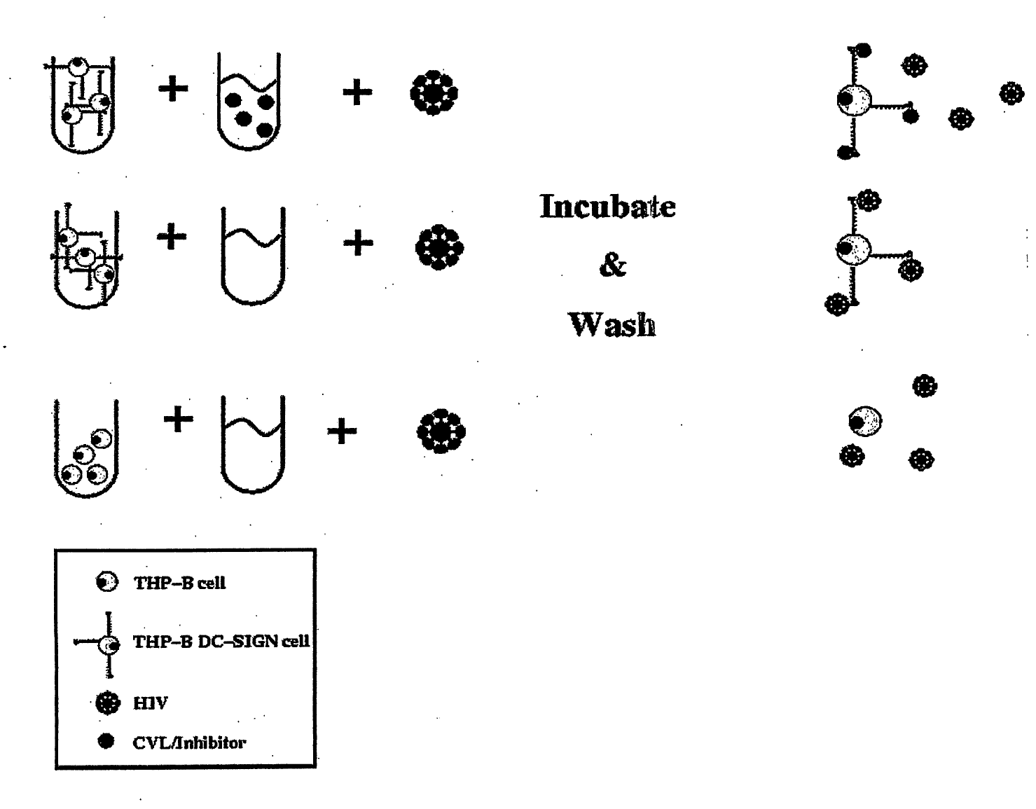

Dilutions of CVL samples were prepared and added to a monocytic cell line that had been transformed to express DC-SIGN (THP DC-SIGN). After a one hour incubation, HIV (either M- or T-tropic) was added to the reaction. The cells were incubated with the virus and the CVL for another hour. Unbound virus was removed by washing the cells three times in medium containing 1% Fetal Bovine Serum (FBS). An HIV p24 antigen capture assay was then carried out to determine the amount of virus that remained bound to the cells. Inhibition of binding was determined as a percentage of a positive control that received a “mock” CVL of saline. The experimental design is shown in FIG. 1.

One sample that had particularly high activity (retaining 50% activity at approximately a 1:300 dilution) was selected for fractional analysis. Size Exclusion High Pressure Liquid Chromatography (SE-HPLC) was performed. SE-HPLC separates substances based solely on their size, and is a typical first step in protein purification. Fifteen fractions were collected from the column and tested for activity. Activity remained intact in the first four fractions, then diminished (with two fractions displaying a possible binding enhancement).

Since the activity was limited to the earlier fractions, the activity was further characterized using spin filters (Microcon) with pore sizes that exclude molecular weights of 10 and 100 kDA. No activity passed through the 10 kDA filter and very little passed through the 100 kDA filter. Almost all the activity stayed on the 100 kDA filter indicating that the molecule or molecules are mostly greater than 100 kDA.

A DEAE Cephacel affinity column was used to further characterize the molecule or molecules. DEAE Cephacel affinity columns bind negatively charged molecules. All the activity passed through the column after two attempts, first using PBS and the second time using 5 mM sodium phosphate, pH 7.4 as binding buffers, and 0.5M NaCl and 0.2M, then 0.4M NaCl as elution buffers, indicating the activity is not a negatively charged protein.

Further characterization was done using a SP sepharose: cation exchange column, which binds positively charged molecules. All the activity again passed through the column on two attempts, using the same two binding and elution buffers noted above, indicating that the activity is not a positively charged protein. Eliminating both positively and negatively charged proteins essentially eliminates all proteins as likely candidate molecules. As a confirmation, active CVL fraction was heated to 95° C. for 5 minutes to denature all proteins. This heating did not destroy any of the activity.

The active CVL fraction was further characterized using a Con A sepharose column. This column binds sugars or glycoproteins with terminal mannose residues. All activity again passed through this column suggesting that terminal mannose residues are not important to the activity. This is relevant, because mannose is a natural ligand of DC-SIGN. It is consistent with the fact that small sugars like mannose, which should have eluted late from the size exclusion column, had no inhibitory activity on HIV binding to DC-SIGN.

The active CVL fraction was further characterized using a Staphylococcus protein A column, which binds IgG. Some activity was retained on the column although the majority was not. Although this contradicts the other findings because it suggests that some activity may be IgG, which is a protein, an alternative explanation is that the molecule shares at least one physical characteristic with IgG that provides binding to Staphylococcus protein A.

Additional characterizations included running the active CVL fraction over Jacalin-agarose beads, which bind IgA. No activity was retained on the beads, indicating the activity is not IgA. Also, the active CVL fraction was subjected to Trypsin-agarose beads, which should digest all protein. This had no effect on the activity of the CVL fractions, again indicating that it is not a protein.

### Example 2 Primary Dendritic Cells Contacted with the Compound are Unable to Present HIV to T Cells

CVLs were collected from high risk and low risk women and heat inactivated. Following centrifugation and filtration the CVL supernatant were tested as follows. THP DC-SIGN cells were exposed to HIV-1BAL in the presence and absence of CVLs at 37° C. for 1 hour and then washed to remove unbound virus. The cells were then washed and lysed. Bound HIV was determined by using a p24 assay on the cellular lysates. The HIV-1-p24 antigen ELISA assay is well known to those of skill in the art. The assay is a twin-site sandwich ELISA and has been described in detail in the art (Moore et al., Science, 250: 1139-1142 (1990) and Moore et al., J. Virol. 65: 852-860, 1991). Briefly, p24 antigen is captured from a detergent lysate of virions onto a polyclonal antibody adsorbed onto a solid phase. Bound p24 is detected with an alkaline phosphatase-conjugated anti-p24 monoclonal antibody and the AMPAK ELISA amplification system. This assay is in routine use throughout USA and Europe for monitoring the rate of HIV production in tissue culture (Patience et al., Methods in Molecular Biology Vol 8: Practical Molecular Virology: The Humana Press Inc., Clifton N.J. pp 131-140.1991). In a typical p24 assay, antibodies, such as D7320 D7330 BC 1071 (described in Weiss et al., Nature 316:69-72, 1985; Weiss et al., Nature 349:374, 1991; Spence et al. J. Gen. Virol., 70:2843-2851, 1989; Ferns et al., AIDS 3:829-834, 1989), and BC 1071-AP, commercially available from Aalto Bio. Reagents Ltd (Dublin, Ireland). The assay may be calibrated using known amounts of purified recombinant p24 (AIDS 4:1125-1131, 1990).

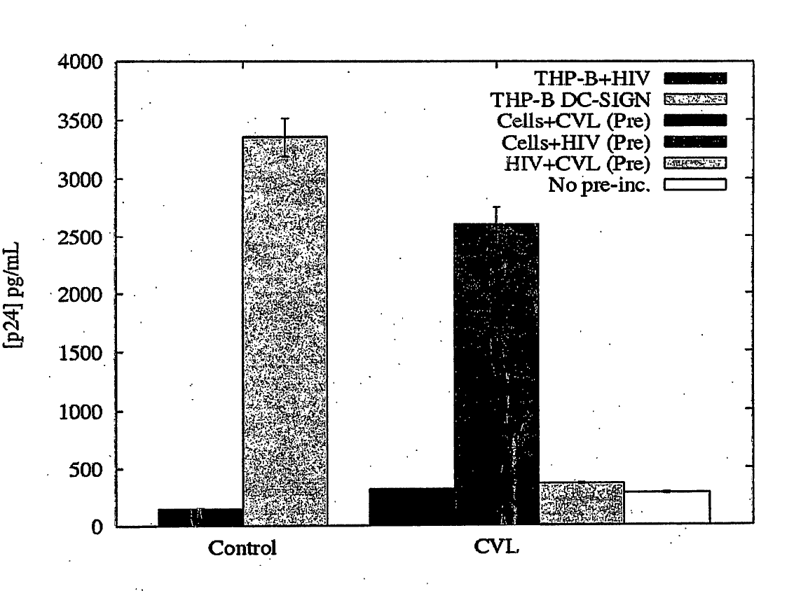

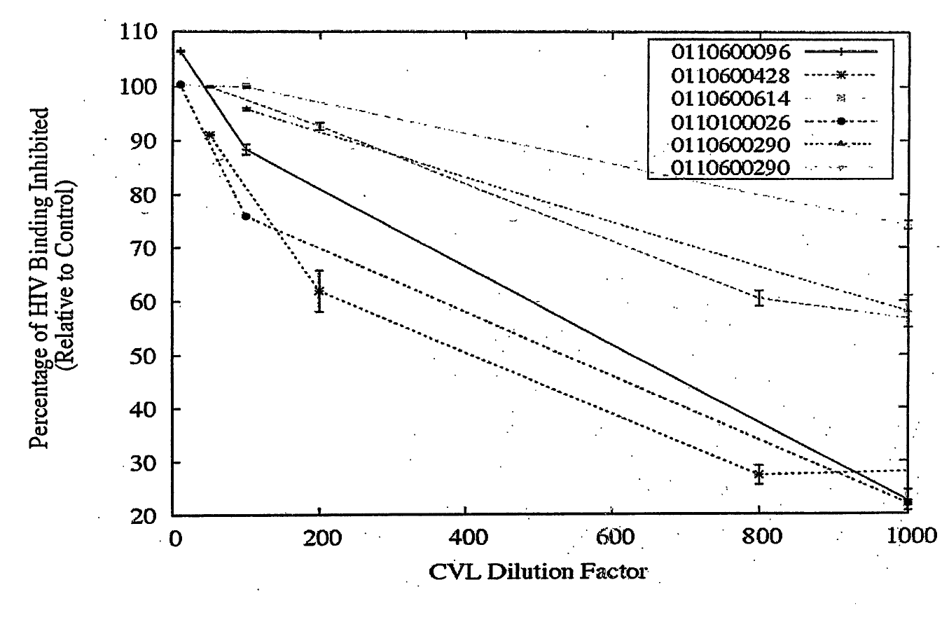

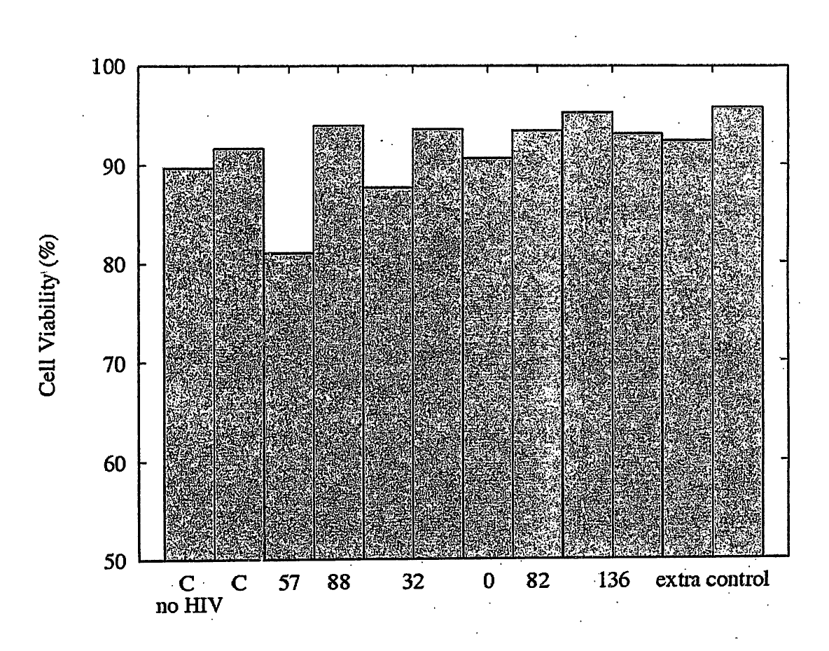

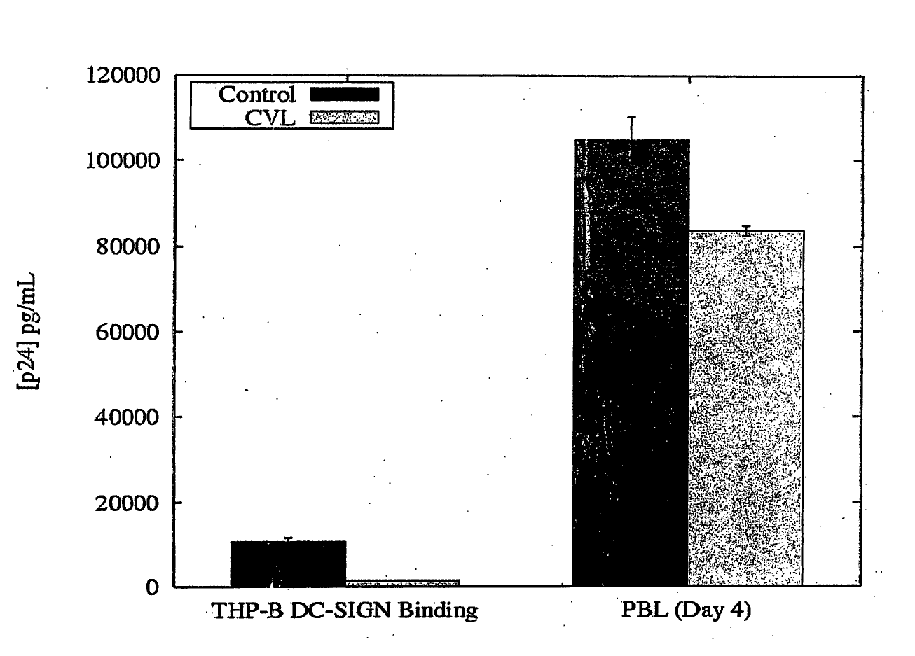

Using a p24 assay on cellular lysates, the percentage inhibition of binding of DC-SIGN to HIV was calculated as a percentage of HIV bound by THP DC-SIGN cells exposed to a mock CVL composed of saline alone. Background and non-specific binding was determined as the amount of HIV bound to TBP cells exposed to a mock CVL. The test CVLs inhibit BAL and MN binding (FIG. 2). A further demonstration of the significant inhibition of HIV binding by CVL is depicted in FIG. 3. A series of dilutions of CVL were performed and showed that the inhibition of binding of HIV to DC was dose-dependent (FIG. 4). A cell viability assay was performed to determine whether the factor from CVL was cytotoxic. As can be seen from FIG. 5, the inhibitor from CVL was not cytotoxic. The effects of viral binding or growth in peripheral blood lymphocytes (PBL) is not affected by CVL. (FIG. 6).

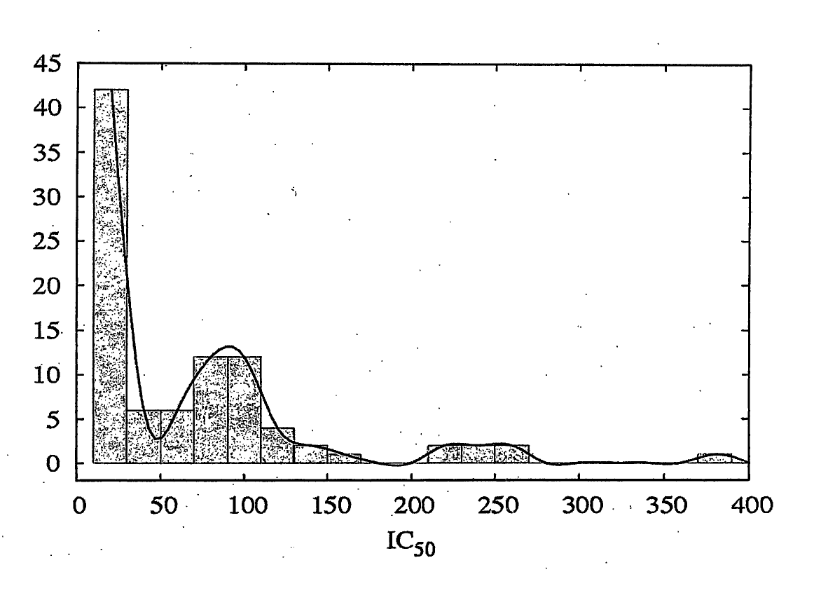

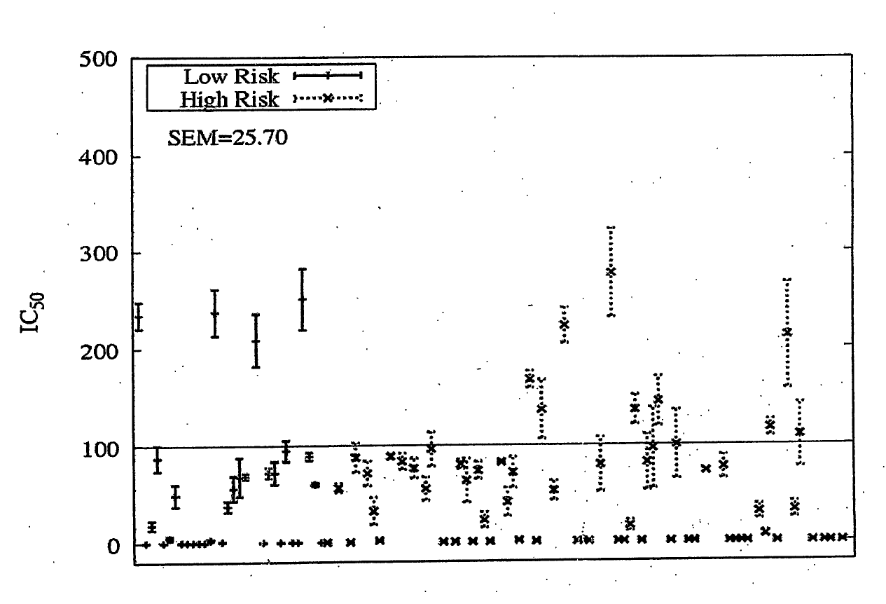

In addition to the inhibition assay results depicted in FIGS. 2-6, the IC50 of the CVL-derived inhibitor was also determined. The activity of each CVL was determined for at least two dilutions and the results were depicted as Dilution factor vs. Binding Inhibition. The slope of line is used to determine the dilution point at which 50% of the HIV bound to DC-SIGN would be blocked. The IC50 values reported is the inverse of that dilution. The IC50 is shown in FIG. 7, and the IC50s at baseline are shown in FIG. 8.

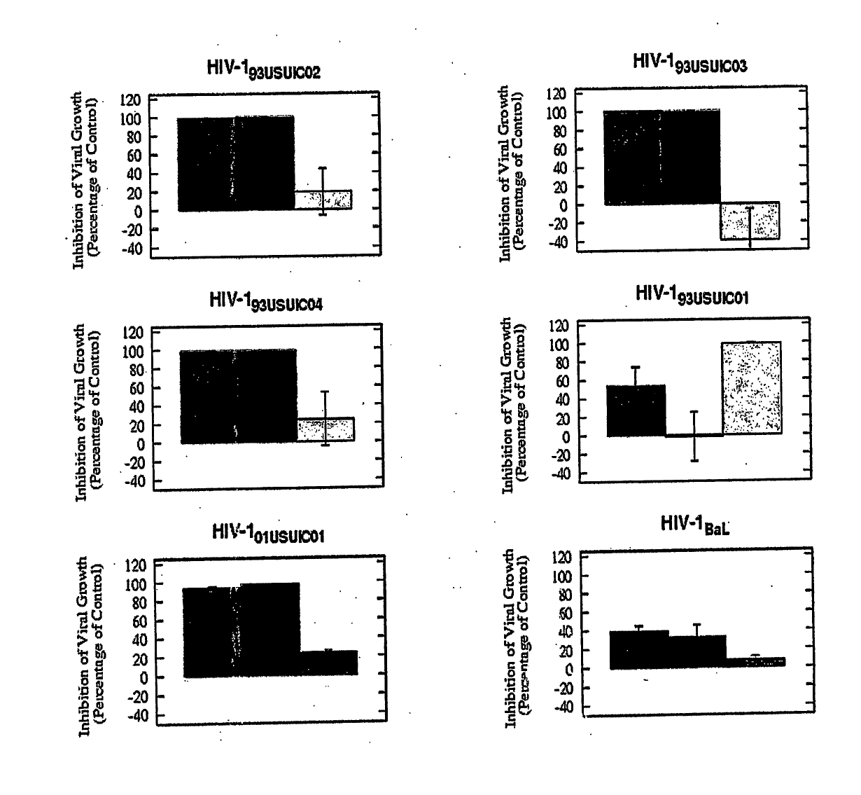

MDDC were exposed to virus (20,000 pg) in the presence and absence of CVL. Unbound virus was removed by washing. Unbound virus was removed by washing and HIV-loaded MDDC cells were added to PHA-stimulated, autologous peripheral blood lymphocytes (PBL) and co-incubated. Supernatants were assayed for p24 on days days 3, 7, and 10 post-initiation of the co-incubation. Similarly, PBMCs from patients were co-cultured with healthy donor PBMCs. Further propagation of the virus was performed using the same donor for all primary isolated and no virus stock was passaged more than three time. The data from these studies are depicted FIG. 11A-F. The identity of the primary isolates in those figures is as follows: 93USUIC01 is a female infected as a result of injection drug use; 93USUIC02 is a male infected through homosexual sex; 93 USUIC03 is a male infected as a result of injection drug use; 93 USUIC04 is a male infected as a result of injection drug use and 01USUIC01 is a female infected as a result of heterosexual sex.

The inhibitor is capable of blocking the binding of most R5 primary isolates to DC. The inhibitor acts to inhibit HIV-DC SIGN interactions and is naturally found in the female genital tract. CVLs containing this inhibitor were able to block most primary HIV isolates from binding to DC and it is believed this inhibitor will contribute to protections from HIV transmission. The presence of this inhibitor is associated with a history of HIV positive sexual partners. This inhibitor may be an inducible host factor produced by women who have HIV positive partners. Without being bound to any particular theory, it is possible that the agent is induced by in response to an inflammatory response. Such an inflammatory response may for example be caused by the immune system in the woman as a response to the traumatized tissue because this agent is produced in those individuals that have frequent intercourse. The inhibitory factor also is positively correlated with the presence of semen in the woman's vaginal tract.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

1. A compound isolated from a cervicovaginal lavage that inhibits binding between dendritic cell-specific ICAM-3 grabbing non-integrin (DC-SIGN) and human immunodeficiency virus (HIV), said compound (a) comprising a carbohydrate; (b) lacking a terminal mannose residue; (c) having a molecular weight of greater than 1100 kDa; (d) retaining function after ten minutes at 95° C.; (d) binding to DC-SIGN; and (e) inhibiting binding of HIV to DC-SIGN.

2. The compound of claim 1, wherein the compound is resistant to trypsin digestion.

3. The compound of claim 1, wherein the compound is susceptible to periodate oxidation.

4. The compound of claim 1, wherein the compound binds to DC-SIGN in a calcium-dependent manner.

5. A composition comprising a compound of claim 1 and a pharmaceutically-acceptable carrier.

6. The composition of claim 5, wherein the composition is formulated for vaginal administration.

7. The composition of claim 5, wherein the composition is formulated for rectal administration.

8. A method of inhibiting binding of HIV to DC-SIGN, said method comprising contacting a cell expressing DC-SIGN on its surface with an amount of a compound of isolated from a cervicovaginal lavage that inhibits binding between dendritic cell-specific ICAM-3 grabbing non-integrin (DC-SIGN) and human immunodeficiency virus (HIV), said compound (a) comprising a carbohydrate; (b) lacking a terminal mannose residue; (c) having a molecular weight of greater than 100 kDa; (d) retaining function after ten minutes at 95° C.; (d) binding to DC-SIGN; and (e) inhibiting binding of HIV to DC-SIGN wherein the amount is effective to inhibit HIV binding to DC-SIGN.

9. The method of claim 8, wherein the cell is contacted in vitro.

10. The method of claim 8, wherein the cell is contacted in vivo.

11. The method of claim 10, wherein a composition of claim 5 is administered to a patient at risk of exposure to HIV.

12. The method of claim 10, wherein a composition of claim 5 is administered to a patient subsequent to exposure to HIV at a location in the patient.

13. The method of claim 12, wherein the composition is administered to the location of exposure.

14. A method of isolating a microbe that produces a compound of any claim 1, said method comprising: (a) testing for a presence of said compound in vaginal fluid of a woman; (b) isolating an organism from the vaginal fluid; and (c) testing the organism for production of the compound.

15. A kit comprising a compound of claim 1.

16. The kit of claim 15, wherein the kit comprises a composition of claim 5.

17. A method of determining the structure of a compound of claim 1, said method comprising isolating the compound from a cervicovaginal lavage and determining the structure of the carbohydrate.

18. A purified and isolated compound which is a carbohydrate which, in nature, is produced in the genital tracts of women that have been exposed to the semen of an HIV positive sexual partner but are HIV negative wherein the compound has a molecular weight greater than 100 kDa, is resistant to trypsin digestion, is resistant to temperatures greater than 80° C., and is an inhibitor of HIV binding to DC-SIGN.

19. The compound of claim 18 in a combination with a pharmaceutically acceptable carrier or diluent.

20. The compound of claim 18, wherein said compound is formulated into a composition for topical administration.

21. A method comprising locally administering to an appropriate region of a human body a pharmaceutically effective amount of a compound of claim 1 or a pharmaceutically acceptable formulation thereof, in an amount effective to reduce transmission of HIV-1 infection or HIV-2 infection.

22. A method of decreasing vaginal transmission of HIV-1 or HIV-2, by vaginal administration of a formulation containing a pharmaceutically effective amount of an inhibitory compound claim 1, wherein the amount is effective to inhibit the binding of HIV to DC-SIGN.

23. The method of claim 22, wherein said vaginal transmission of HIV-1 or HIV-2 is selected from the group consisting of transmission during sexual intercourse or during childbirth.

24. A method of preventing transmission of HIV-1 or HIV-2 in a newborn baby by topically administering to the baby an effective amount of a CVI, derived inhibitor of claim 1, either alone or in combination with a carrier, wherein the amount is effective to inhibit the binding of HIV to DC-SIGN.

25. The method of claim 24, wherein said administration is carried out during childbirth.

26. The methods of claim 24, wherein said administration is carried out immediately after childbirth

27. A contraceptive device wherein the device has applied thereto an effective amount of a compound of claim 1, or a pharmaceutically acceptable salt thereof, in an amount effective to inhibit the binding of HIV to DC-SIGN.

28. A pessary or tampon for vaginal administration, wherein the tampon or pessary comprises as an active ingredient, a pharmaceutically effective amount of a compound described herein or a pharmaceutically acceptable salt thereof and one or more pharmaceutically acceptable carriers or excipients, wherein said amount is sufficient to inhibit the binding of HIV to DC-SIGN.

29. (canceled)
